(19) United States
(12) Patent Application Publication
(10) Pub. No.: US 2024/0041610 A1
(43) Pub. Date: Feb. 8, 2024

(54) ORTHOPEDIC IMPLANTS AND METHODS

(71) Applicant: Paragon 28, Inc., Englewood, CO (US)

(72) Inventors: Andrew Julian GOLDBERG, Radlett (GB); Kevin Alan DODD, Round Rock, TX (US); Mathew Richard ANDERLE, Greenwood Village, CO (US); Michael Edward CARROLL, Memphis, TN (US); Richard Matthew OBERT, Poway, CA (US); Gregory Charles MARIK, Germantown, TN (US)

(73) Assignee: Paragon 28, Inc., Englewood, CO (US)

(21) Appl. No.: 18/477,170

(22) Filed: Sep. 28, 2023

Related U.S. Application Data
(63) Continuation of application No. PCT/US22/71438, filed on Mar. 30, 2022.
(60) Provisional application No. 63/167,965, filed on Mar. 30, 2021.

(51) Int. Cl. A61F 2/42

(52) U.S. Cl. CPC A61F 2/4202; A61F 2002/4207; A61F 2002/4629

(57) ABSTRACT

A subtalar joint implant system that includes a first implant component and a second implant component is disclosed herein. An alternative embodiment of a subtalar joint implant system is disclosed and includes a first component that has threads disposed on at least a portion of an exterior surface of the first component and a second component having threads disposed on at least a portion of the exterior surface of the second component. The first and second components are each configured to couple with at least a portion of a talus and a calcaneum of a patient with the first component being configured to couple with at least a portion of a posterior facet of the calcaneum of the patient and the second component being configured to couple with at least a portion of a middle facet of the calcaneum of the patient.

### CROSS-REFERENCE TO RELATED APPLICATIONS

This application is a bypass continuation of PCT Application No. PCT/US2022/071438, filed Mar. 30, 2022, and entitled “Orthopedic Implants and Methods,” which claims priority benefit under 35 U.S.C. § 119(e) of U.S. Provisional Application No. 63/167,965 filed Mar. 30, 2021, and entitled “Orthopedic Implants and Methods,” which are incorporated herein by reference in their entirety.

### TECHNICAL FIELD

The present disclosure relates to implants, devices, and methods associated with performing arthroplasty procedures. The present disclosure relates to podiatric and orthopedic implants and surgery related to arthroplasty, arthrodesis, and/or arthroeresis of joints in the foot/ankle and/or procedures incorporating surrounding bones/soft tissue. More specifically, but not exclusively, the present disclosure relates to implants, devices, and methods relating to arthroplasty of the subtalar joint.

### BACKGROUND OF THE INVENTION

Many currently available implants, devices, and methods for addressing joint trauma (acute and chronic, e.g., defect, gradual deterioration, etc.) do not completely address the needs of patients. Additionally, many currently available implants, devices, and methods for addressing joint trauma fail to account for properties of joint anatomy and associated mechanical and kinematic movement patterns/capabilities.

### SUMMARY OF THE INVENTION

The present disclosure is directed toward implants, devices, and related methods for maintaining, correcting and/or resurfacing joint surfaces.

A first aspect of the present disclosure includes a subtalar joint implant system. The subtalar joint implant system includes a first implant component, and a second implant component.

According to the first aspect of the present disclosure, the first implant component includes a top portion and a bottom portion. The top and bottom portions are coupled to one another and are configured about a common longitudinal axis.

According to the first aspect of the present disclosure, the subtalar joint implant system includes the top portion and the bottom portion of the first implant component threadably couplable with one another.

According to the first aspect of the present disclosure, the subtalar joint implant system includes the top portion of the first implant component having an engagement feature.

According to the first aspect of the present disclosure, the engagement feature includes an interface which is configured to engage with one or more instruments configured to facilitate implantation of the implant.

According to the first aspect of the present disclosure, the bottom portion includes a threading disposed on at least a portion of an outer surface thereof, and an engagement feature disposed on an end portion of the bottom portion. The engagement feature is configured to engage with one or more instruments configured to facilitate implantation of the first portion of the implant.

According to the first aspect of the present disclosure, the top portion of the first implant component is configured to be implanted in at least a portion of a talus of a patient.

According to the first aspect of the present disclosure, the bottom portion of the first implant component is configured to be implanted in at least a portion of a calcaneum of the patient.

According to the first aspect of the present disclosure, the bottom portion of the first implant component is configured to be implanted in at least a portion of a posterior facet of the calcaneum of the patient.

According to the first aspect of the present disclosure, the second implant component includes a top portion and a bottom portion. The top and bottom portions are coupled with one another and are configured about a common longitudinal axis.

According to the first aspect of the present disclosure, the top portion and the bottom portion of the second implant component are threadably couplable with one another.

According to the first aspect of the present disclosure, the top portion of the second implant component includes an engagement feature.

According to the first aspect of the present disclosure, the engagement feature includes an interface which is configured to engage with one or more instruments configured to facilitate implantation of the implant.

According to the first aspect of the present disclosure, the bottom portion of the second implant component includes a threading disposed on at least a portion of an outer surface thereof and an engagement feature disposed on an end portion of the bottom portion. The engagement feature is configured to engage with one or more instruments configured to facilitate implantation of the second portion of the implant.

According to the first aspect of the present disclosure, the top portion of the second implant component is configured to be implanted in at least a portion of a talus of a patient.

According to the first aspect of the present disclosure, the bottom portion of the second implant component is configured to be implanted in at least a portion of a calcaneum of the patient.

According to the first aspect of the present disclosure, the bottom portion of the second implant component is configured to be implanted in at least a portion of a middle facet of the calcaneum of the patient.

A second aspect of the present disclosure includes a subtalar implant system. The system includes a first component having a top portion and a bottom portion coupled with the top portion and configured about a first common longitudinal axis. The system also includes a second component having a top portion and a bottom portion coupled with the top portion and configured about a second common longitudinal axis. The first and second components are each configured to couple with at least a portion of a calcaneum and a talus of a patient.

According to the second aspect of the present disclosure, the first component is configured to couple with at least a portion of a posterior facet of the calcaneum of the patient and the second component is configured to couple with at least a portion of a middle facet of the calcaneum of the patient.

A third aspect of the present disclosure includes a subtalar implant system. The system includes a first component having a threading disposed on at least a portion of an exterior surface thereof, and a second component having a threading disposed on at least a portion of an exterior surface thereof. The first and second components are each configured to couple with at least a portion of a talus and a calcaneum of a patient. Further, the first component is configured to couple with at least a portion of a posterior facet of the calcaneum of the patient and the second component is configured to couple with at least a portion of a middle facet of the calcaneum of the patient.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a top view of a calcaneus shown in the transverse plane, in accordance with the present disclosure;

FIG. 2 is a bottom view of a talus shown in the transverse plane, in accordance with the present disclosure;

FIG. 3 is a top view of skeletal structures of the midfoot and hindfoot, in accordance with the present disclosure;

FIG. 4 is a top view of skeletal structures of the midfoot and hindfoot, in accordance with the present disclosure;

FIG. 5 is a top view of skeletal structures of the midfoot and hindfoot, in accordance with the present disclosure;

FIG. 6 is a top view of skeletal structures of the midfoot and hindfoot, in accordance with the present disclosure;

FIG. 7 is a bottom perspective view of an implant system, in accordance with the present disclosure;

FIG. 8 is a rear perspective view of the implant system of FIG. 7, in accordance with the present disclosure;

FIG. 9 is a top view of the implant system of FIG. 7, in accordance with the present disclosure;

FIG. 10 is a rear elevated perspective view of the implant system of FIG. 7, in accordance with the present disclosure;

FIG. 11 is a rear lower perspective view of the implant system of FIG. 7, with a portion of the bone being shown as transparent to show the implanted system, in accordance with the present disclosure;

FIG. 12 is a front lower perspective view of the implant system of FIG. 7, in accordance with the present disclosure;

FIG. 13 is a bottom view of the implant system of FIG. 7, in accordance with the present disclosure;

FIG. 14 is a side view of the implant system of FIG. 7, with a portion of the bone being shown as transparent to show the implanted system, in accordance with the present disclosure;

FIG. 15 is a rear view of the implant system of FIG. 7, with a portion of the bone being shown as transparent to show the implanted system, in accordance with the present disclosure;

FIG. 16 is a top view of an implant system, in accordance with the present disclosure;

FIG. 17 is a bottom view of the implant system of FIG. 16, in accordance with the present disclosure;

FIG. 18 is a front view of the implant system of FIG. 16, in accordance with the present disclosure;

FIG. 19 is a side view of the implant system of FIG. 16, in accordance with the present disclosure;

FIG. 20 is a rear view of the implant system of FIG. 16, in accordance with the present disclosure;

FIG. 21 is a side view of the implant system of FIG. 16, in accordance with the present disclosure;

FIG. 22 is an elevated rear perspective view of the implant system of FIG. 16, in accordance with the present disclosure;

FIG. 23 is an elevated front perspective view of the implant system of FIG. 16, in accordance with the present disclosure;

FIG. 24 is an elevated perspective view of an implant system, in accordance with the present disclosure;

FIG. 25 is an elevated front perspective view of the implant system of FIG. 24, in accordance with the present disclosure;

FIG. 26 is a side view of the implant system of FIG. 24, in accordance with the present disclosure;

FIG. 27 is a rear view of the implant system of FIG. 24, in accordance with the present disclosure;

FIG. 28 is a side view of the implant system of FIG. 24, in accordance with the present disclosure;

FIG. 29 is a front elevated perspective view of an implant system, in accordance with the present disclosure;

FIG. 30 is a rear elevated perspective view of the implant system of FIG. 29, in accordance with the present disclosure;

FIG. 31 is a side elevated perspective view of the implant system of FIG. 29, in accordance with the present disclosure;

FIG. 32 is a top view of the implant system of FIG. 29, in accordance with the present disclosure;

FIG. 33 is a front perspective view of an implant system, in accordance with the present disclosure;

FIG. 34 is a top view of the implant system of FIG. 33, in accordance with the present disclosure;

FIG. 35 is a front perspective view of an alternative embodiment of the implant system of FIG. 33, in accordance with the present disclosure;

FIG. 36 is a top view of an alternative embodiment of the implant system of FIG. 33, in accordance with the present disclosure;

FIG. 37 is a perspective view of an implant system, in accordance with the present disclosure; and

FIG. 38 is a top view of the implant system of FIG. 37, in accordance with the present disclosure.

### DETAILED DESCRIPTION OF THE INVENTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers to the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in U.S. Pat. No. 10,117,749, issued on Nov. 6, 2018 and entitled “Subtalar Joint Implant”; European Patent No. 3756626 issued on Dec. 30, 2020 and entitled “Subtalar Joint Implant”, European Patent Application No. 15770960.1A filed on Jul. 15, 2020 and entitled “Subtalar Joint Implant”; U.S. patent application Ser. No. 17/653,029, filed on Mar. 1, 2022 and entitled “Methods for Performing an Arthroplasty of the Subtalar Joint”; and U.S. Provisional Patent Application No. 63/155,100 filed on Apr. 2, 2021 and entitled “Systems and Methods for Controlled Facet Repositioning in the Calcaneus”; which are all hereby incorporated herein by reference in their entireties.

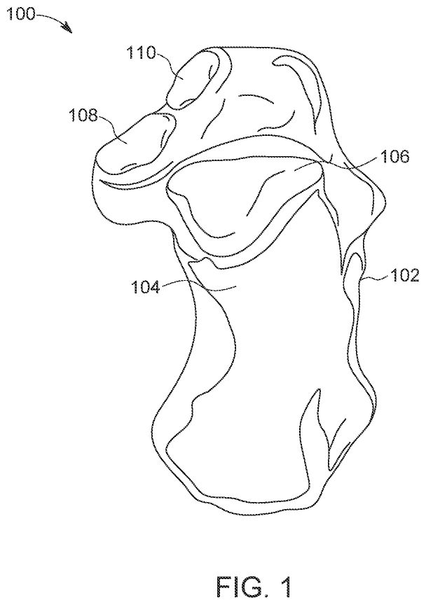

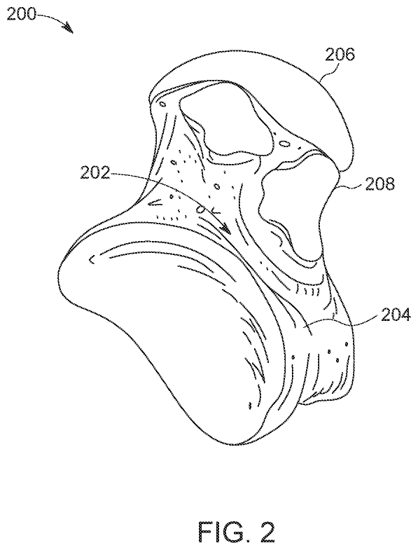

Referring to the drawings, wherein like reference numerals are used to indicate like or analogous components throughout the several views, and with particular reference to FIGS. 1-2, there is illustrated an exemplary embodiment of a right calcaneum (e.g., calcaneus) 100 shown from a superior view and a right talus 200 shown from an inferior view. The calcaneum 100 and the talus 200 are bones of the foot/ankle and two bones of the subtalar joint which is disposed substantially inferior (e.g., distal) relative to the ankle. According to some research and corresponding literature, the subtalar joint may be considered two joints based on one or more facets disposed on the calcaneum 100, which are shown with reference to FIG. 1 and described subsequently herein. These two joints include a first joint (the subtalar joint) including the posterior facet of the calcaneum 100, and a second joint (the talocalcaneonavicular joint) including the middle and/or anterior facet as well as the talonavicular joint (including the talus 200 and the navicular (not shown). However, for the purpose of this disclosure the subtalar joint is considered to include at least the calcaneum 100 (including the posterior, middle, and anterior facets), the talus 200, and the soft tissue disposed therebetween. The subtalar joint, which is positioned superior relative to the calcaneum 100 and inferior relative to the talus 200 is configured to permit/enable movement. Typically, the subtalar joint permits movement that includes at least inversion and eversion of the foot, where inversion is defined as a movement that causes the soles of the feet to face inwards (e.g., in a medial direction) and eversion is defined as a movement that causes the soles of the feet to face outwards (e.g., laterally). Further, inversion and eversion can include external and internal rotation, respectively, of the talus 200 relative to (e.g., on) the calcaneum 100. The calcaneum 100 and the talus 200 (and components thereof) are shown and described herein so as to provide context for various steps of the subsequently disclosed method of performing arthroplasty of the subtalar joint. Furthermore, it should be noted that the calcaneum 100 and the talus 200 as shown correspond to such bones found in the right foot of a human and that the structures shown and described herein may have different sizing and/or orientation with respect to the left foot of a human (but can be reasonably assumed to be equal and opposite) in the medial-lateral directions. It should be understood that the calcaneum 100 and the talus 200 may not be representative of the geometry of such bones of any/all humans/patients but are shown herein to be representative of the general geometry and features of the bones. For example, trauma, bone deformity, arthritis and other conditions can alter the geometry of such bones. As referred to herein, arthroplasty is defined as surgical reconstruction and/or joint replacement of a joint (in the context of this disclosure, the subtalar joint). It should be known that both the calcaneum 100 and the talus 200 may include other geometric features, interfaces with other portions of anatomy, and articulations in addition to those discussed herein.

The calcaneum 100 is shown to include a body 102 which includes a top surface 104. The top surface 104 of the calcaneum 100 may substantially include a superior surface of the calcaneum 100 and form at least a portion of an inferior portion and/or defining surface of the subtalar joint. In some aspects, the top surface 104 interfaces with cartilage positioned between the calcaneum 100 and the talus 200 (e.g., the subtalar joint) that may be removed in performing a subtalar joint arthroplasty. In some aspects, the top surface 104 may become one of two interfacing surfaces of a subtalar joint after arthroplasty is performed (e.g., a surface that interfaces with a subtalar joint implant). The top surface 104 may have various geometries depending on the patient and the condition of the calcaneum 100 (e.g., trauma, arthritis, deformity, etc.). As shown, the top surface 104 includes a posterior facet 106, a middle facet 108, and an anterior facet 110 as shown in FIG. 1. In some aspects, one or more of the posterior facet 106, the middle facet 108 and the anterior facet 110 may be less distinguishable than shown on the calcaneum 100 in FIG. 1. For example, in some aspects the middle facet 108 and the anterior facet 110 may be positioned closer to one another than shown in FIG. 1 such that the middle facet 108 and the anterior facet 110 overlap and/or abut one another (e.g., there is not a gap positioned anterior-lateral the middle facet 108). In some aspects, the calcaneum body 102, the posterior facet 206, the middle facet 108, the anterior facet 110 (as well as other components of the top surface 204 and the calcaneum 100) may be referenced as anthropometric markers (e.g., landscape markers, identifiers, etc.) in order to analyze movement and/or other kinematic properties of the calcaneum 100 and/or surrounding joints. It should also be understood that one or more events or conditions (e.g., trauma, arthritis, deformity, etc.) may alter the calcaneum 100 of a patient such that one or more of the features described has an altered geometry, is damaged, or is not present on the calcaneum 100 of a patient.

The talus 200 is shown to include a body 202 and a bottom surface 204. The top surface 204 of the talus 200 may substantially include an inferior surface of the talus 200 and form at least a portion of a superior portion and/or defining surface of the subtalar joint. In some aspects, the bottom surface 204 interfaces with cartilage positioned between the calcaneum 100 and the talus 200 (e.g., the subtalar joint) that may be removed in performing a subtalar joint arthroplasty. In performing an arthroplasty of the subtalar joint, at least a portion of subcortical bone may be removed in order to place one or more components of an implant so as to promote maximum stability of the joint as well as other surrounding joints and structures. In some aspects, the bottom surface 204 may become one of two interfacing surfaces of a subtalar joint after arthroplasty is performed (e.g., a surface that interfaces with a subtalar joint implant). The bottom surface 204 may have various geometries depending on the patient and the condition of the calcaneum 100 (e.g., trauma, arthritis, deformity, etc.). The talus 200 is further shown to include a talar head 206 positioned at an anterior portion of the talus 200, with a talar neck 208 extending between the body 202 and the talar head 206. In some aspects, the talar body 202, the talar head 206, and the talar neck 208 (as well as other components of the bottom surface 204 and the talus 200) may be referenced as anthropometric markers (e.g., landscape markers, identifiers, etc.)) in order to analyze movement and/or other kinematic properties of the talus 200 and/or surrounding joints.

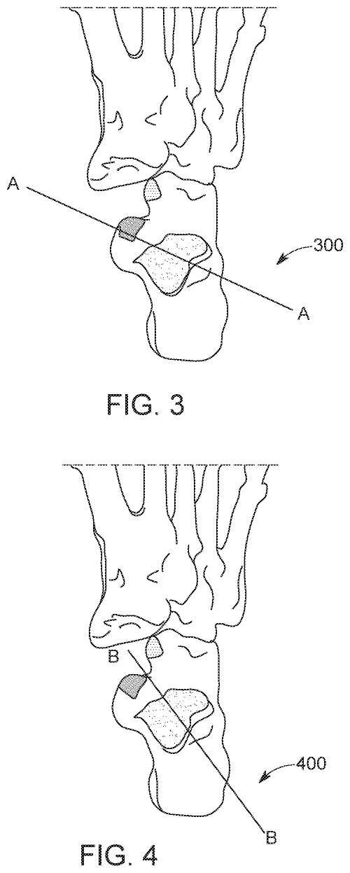

Referring to FIG. 3, an axis 300 is shown to extend along the line A-A from a point at or near (or through) a proximal portion of the middle facet to a portion at or near (or through) the posterior facet. The axis 300 may correspond to an axis of the calcaneum (e.g., an axis about which the calcaneum moves with respect to the talus). The axis 300 may correspond to a best fit cylinder created through the posterior facet and the articular geometry thereof. The axis 300 may extend from a medial prominent apex of the middle facet passing through a prominent point on the medial portion of the posterior facet and terminating near a prominent point on the middle facet.

Referring to FIG. 4, an axis 400 is shown to extend along the line B-B from a point at or near (or through) a distal portion of the middle facet to a portion at or near (or through) the posterior facet. The axis 400 may correspond to an axis of the calcaneum (e.g., an axis about which the calcaneum moves with respect to the talus). The axis 400 may correspond to a best fit cylinder through the articular geometry of the posterior facet. The axis 400 may extend between a medial and posterior prominent apex of the posterior facet to and/or through and/or near a central articulating point within an acetabulum pedis defined by a talar navicular joint, the middle and anterior facets, and a spring ligament complex.

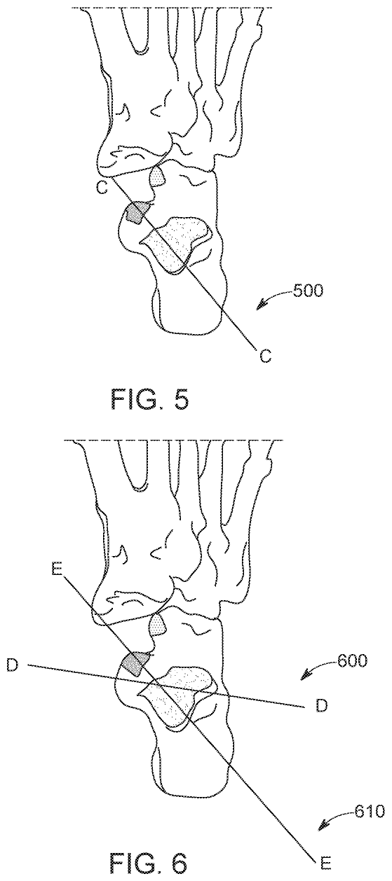

Referring to FIG. 5, an axis 500 is shown to extend along the line C-C from a point at or near (or through) a distal portion of the middle facet to a portion at or near (or through) the calcaneum. The axis 500 may also correspond to an axis of the calcaneum (e.g., an axis about which the calcaneum moves with respect to the talus). The axis 500 may correspond to one or more features/components of the articular geometry of the posterior facet. The axis 500 may extend between a posterior prominent apex of the posterior facet and extend to/pass through a prominent point on the medial portion of the posterior facet and terminating near the center of the middle facet.

Referring to FIG. 6, an axis 600 is shown to extend along the line D-D and an axis 610 is shown to extend along the line E-E. The axes 600, 610 may correspond to an axes of the calcaneum (e.g., an axis about which the calcaneum moves with respect to the talus). The axes 600, 610 may correspond to the articular geometry of the posterior facet. The axes 600, 610 may rotate during activity about an area near the interosseous ligament or the medial prominent aspect of the posterior facet. The axes 600, 610 may also correspond to a heel strike axis and a stance to toe-off axis, about which movement of the calcaneum, talus, and/or subtalar joint may occur.

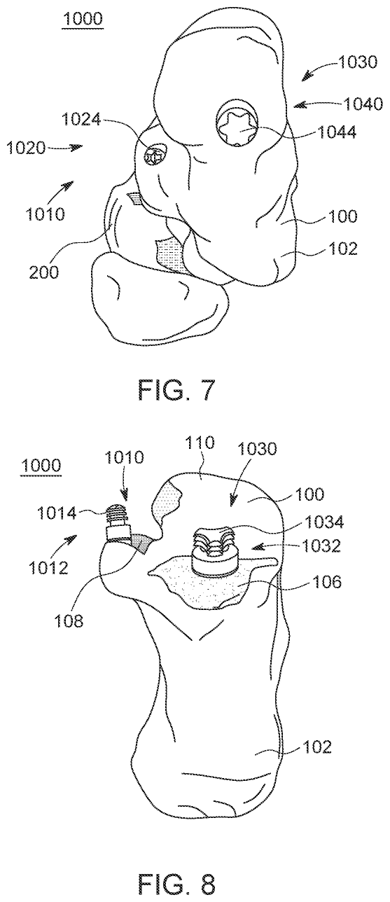

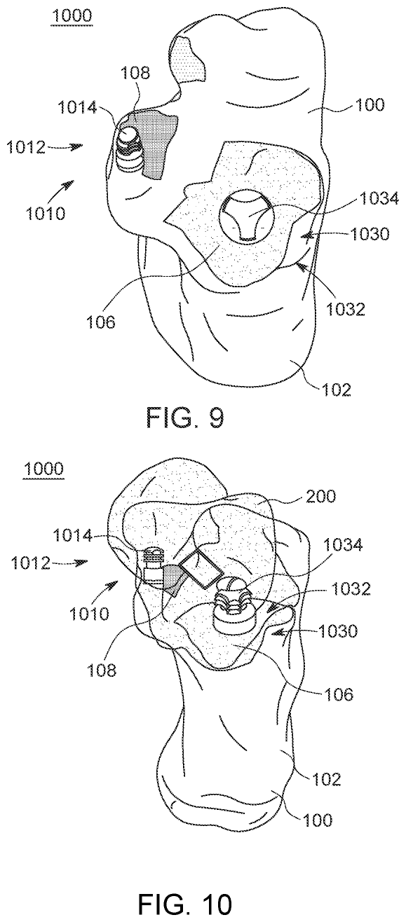

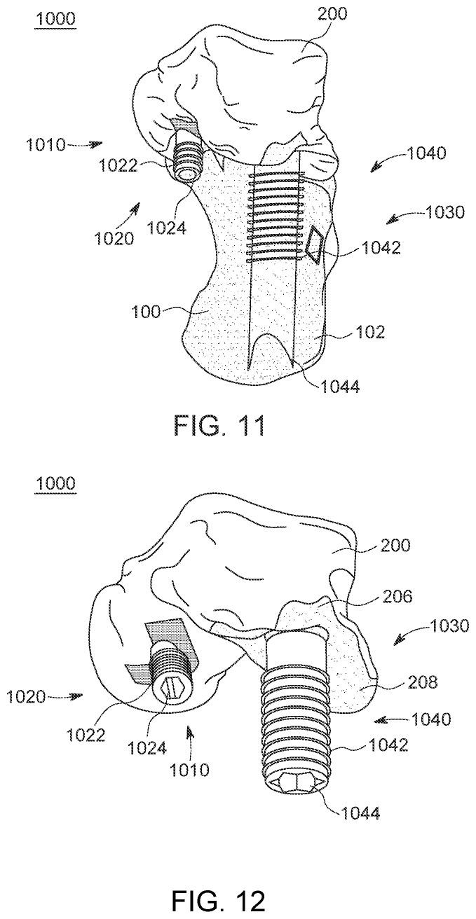

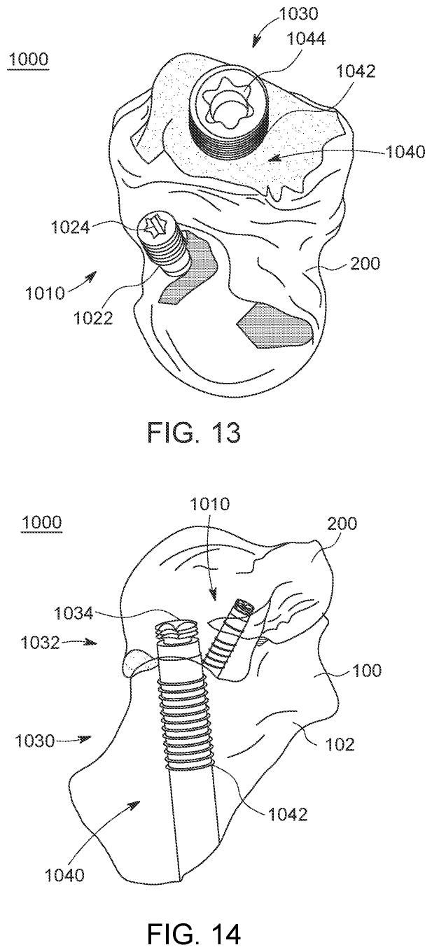

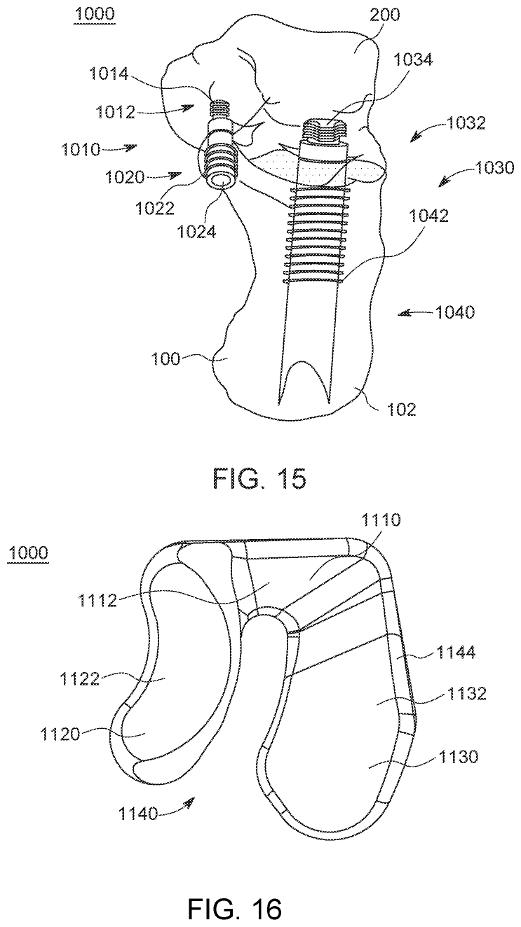

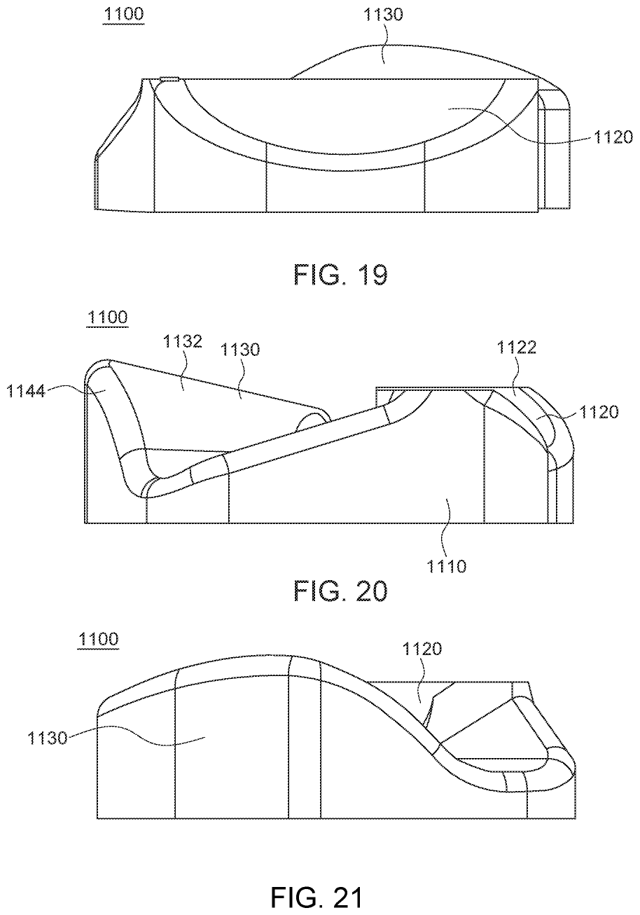

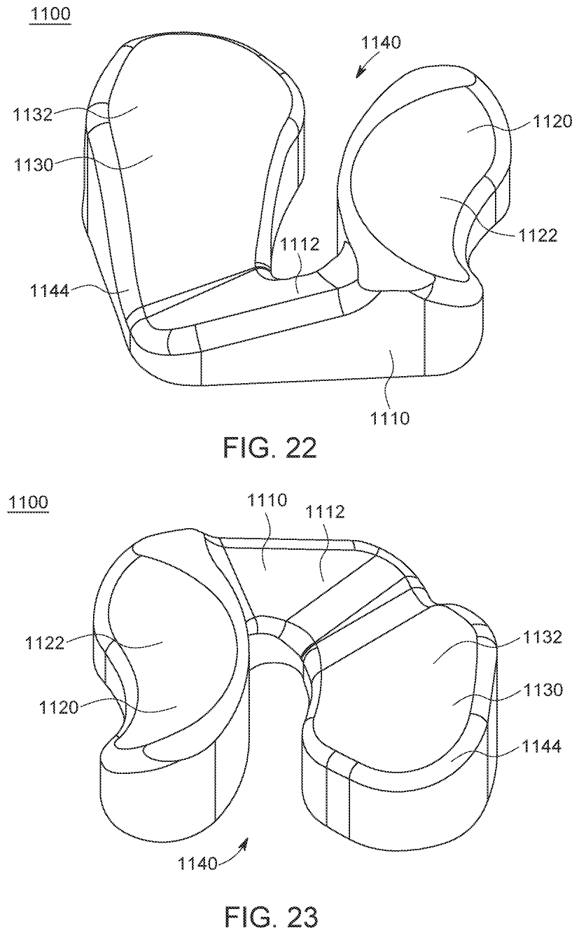

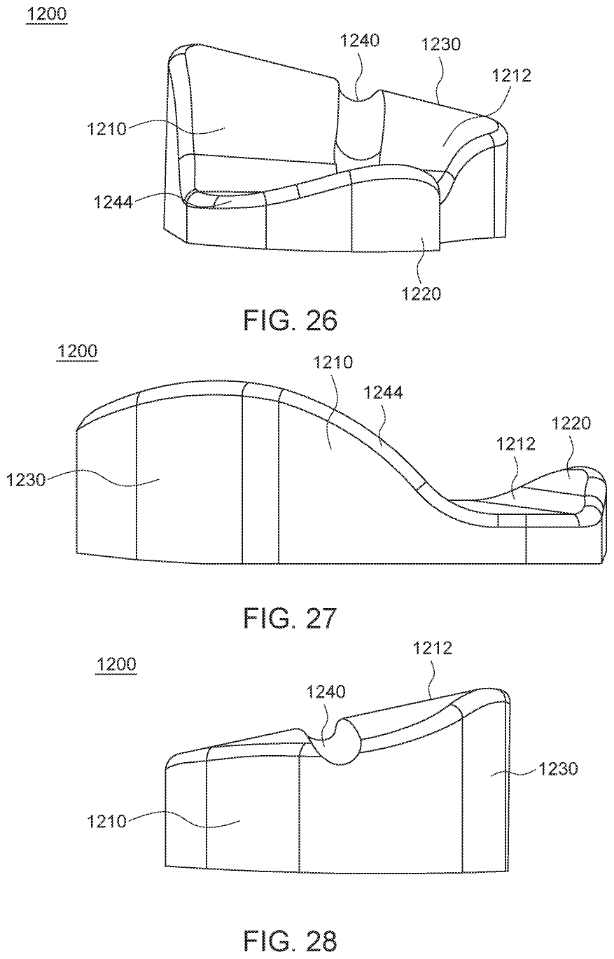

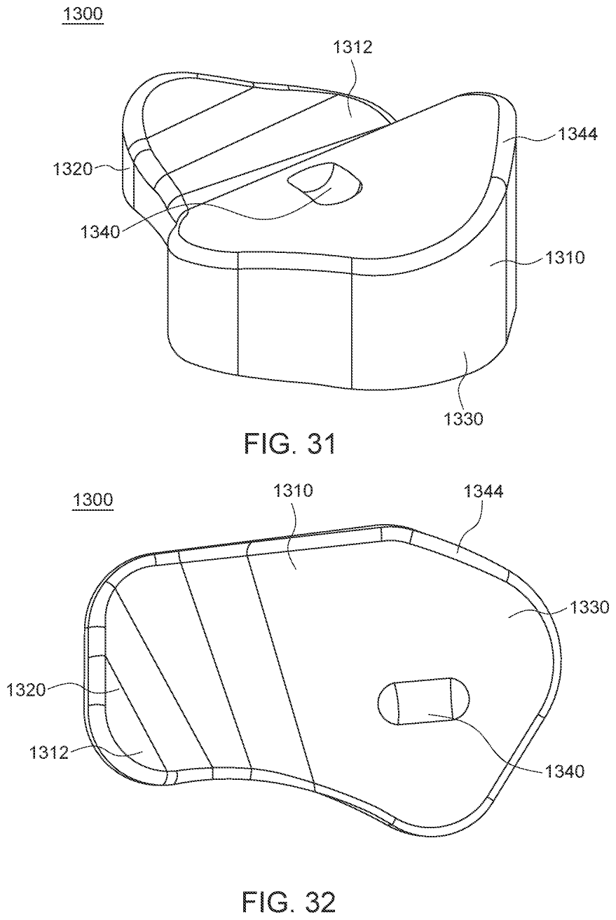

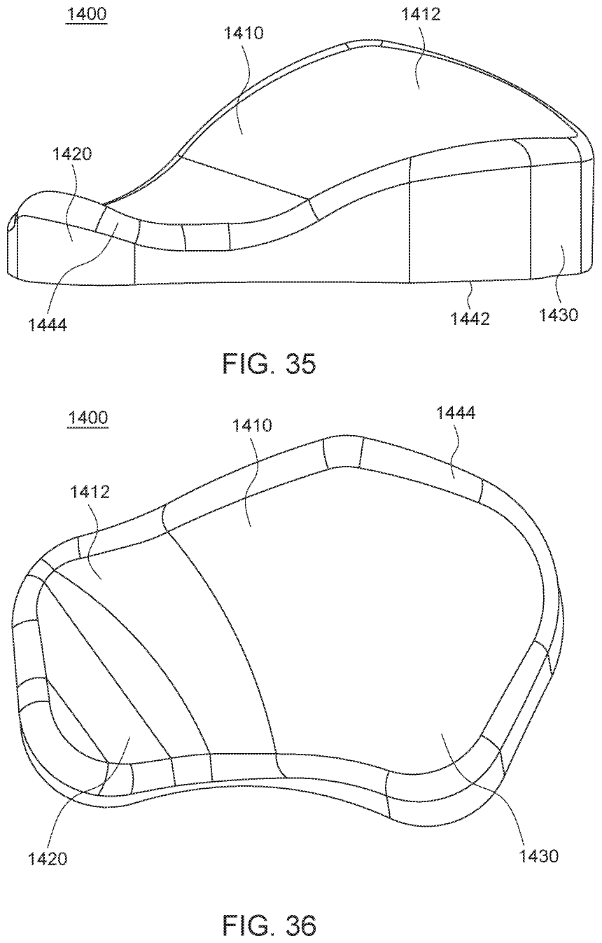

Referring now to FIGS. 7-38, various implant systems are shown. In some aspects, the implant system may be implemented in conjunction with other components (e.g., implants, articulating components, additional aspects of the implant system configured to interface with the components, etc.). The implant systems and components thereof shown and described herein may be configured to be implanted near or adjacent to the subtalar joint and/or substantially adjacent any corresponding musculoskeletal structures. In some aspects, one or more aspects of the implant system may include one or more features (e.g., components, contours, geometries, etc.) configured to be implanted relative to the geometric landmarks shown and described with reference to FIGS. 1-6 herein. For example, a component of an implant system shown and described herein may be configured to be centered about one or more of the axes 300, 400, 500, 600 and/or 610. It should be understood that any of the implant systems shown and described herein may be configured such that a component of one or more systems may be implemented in conjunction with one or more components of one or more of the other disclosed implant systems, including but not limited to those shown, described, and incorporated by reference herein.

Referring now to FIGS. 7-15, an implant system 1000 is shown, according to an exemplary embodiment. In some aspects, the system 1000 may be a subtalar joint arthroplasty system or implant, arthrodesis system, arthroeresis system, or other systems configured to be implanted at, near, adjacent to, or across at least a portion of the subtalar joint. The system 1000 is shown to include multiple components, where at least a portion of one or both of the components may be configured to contact or otherwise engage at least one of the calcaneum 100 and/or the talus 200. In some aspects, the calcaneum 100 and/or the talus 200 may be prepared prior to implantation of the system 1000 (and/or components thereof). For example, in some aspects, one or more axes in the calcaneum 100 and/or the talus 200 may be identified with the calcaneum 100 and/or the talus 200 subsequently prepared according to (e.g., along, adjacent to, or otherwise relative to) the axis or axes. In some aspects, preparation of the calcaneum 100 and/or talus 200 may include creation of a bore or other volume (e.g., one or more; using a drill or other common orthopedic surgical instrument) such that the bore or volume is created about an axis. The aforementioned bore or volume may be configured in the calcaneum 100, the talus 200, or may span both anatomical structures. In some aspects, one or more components of the system 1000 may then be implanted at least partially within the bore or volume.

The implant system 1000 is shown to include a first component 1010 and a second component 1030 which, as shown, are separate components (e.g., not integral or coupled with one another), according to an exemplary embodiment. The first component 1010 is shown to include a substantially cylindrical geometry along the majority of the first component 1010. The first component is further shown to include a top portion 1012 and a bottom portion 1020 which, similar to the first component 1010 as a whole, each include a substantially cylindrical geometry along the majority thereof. In some aspects, the top portion 1012 and the bottom portion 1020 may be releasably couplable with one another (e.g., via an internal threading or other coupling mechanism). Further, in some aspects, the top portion 1012 and the bottom portion 1020 may be integral with one another. Both the top portion 1012 and the bottom portion 1030, when coupled (or integral), are shown to be disposed about a common longitudinal axis (e.g., an axis extending through the cylindrical geometry of both components. In some aspects, one or both of the top portion 1012 and/or the bottom portion 1020 may include a full or partial cannulation extending along some or all of the common longitudinal axis of the components.

The top portion 1012 is shown to include an engagement feature 1014 on an end thereof opposite the bottom portion 1020. The engagement feature 1014 may be configured to interface with one or more of various instruments either via common engagement mechanisms (e.g., tri-lobe, hex-lobe, Phillips, etc.) or via a specialized engagement mechanism configured to facilitate engagement with an instrument specific to the first component 1010. In some aspects, the engagement feature 1014 may have a lesser lateral diameter than other portions of the top portion 1012, although components of the engagement feature 1014 may extend in a lateral direction (e.g., substantially radially) such that a portion of the engagement feature includes a lateral dimension equal to or greater than other portions of the top portion 1012 along the longitudinal axis. Further, in some aspects the engagement feature 1014 may include external threading (which may be partial or intermittent threading, e.g., threading that does not continue circumferentially about the top portion 1012) or other additional/alternate engagement elements also configured to engage either instruments incorporated in conjunction with the system 1000 or, in some aspects, configured to aid in coupling with the calcaneum 100 and/or the talus 200. The top portion 1012 may also include additional threading (not shown) which may be either internal or external and configured to facilitate coupling with the bottom portion 1020.

The bottom portion 1020, as shown, may include a greater lateral dimension (e.g., diameter) than some or all of the top portion 1012. The bottom portion 1020 is shown to include threading 1022 disposed about at least a portion of an exterior surface thereof, as well as an interface 1024 disposed at an end of the bottom portion 1020 substantially opposite the top portion 1012. The threading 1022 may be the same as and/or similar to threading common to orthopedic implants and/or screws (e.g., considered to interface with various different types/portions of bone, for example those that exist at various portions of the calcaneum 100 and/or the talus 200). The interface 1024 is shown to be set in the end portion of the bottom portion 1020 and, in some aspects, may form a depression that extends substantially into at least a portion of the cylindrical geometry of the bottom portion 1020. The interface 1024 may include an interface common to orthopedic instruments, for example a hex-lobe, torxx, Phillips, or other interface, or the interface 1024 may include an alternate interface configured specifically to interface with one or more instruments specific to the system 1000. Additionally, the bottom portion 1020 may also include additional threading (not shown) which may be either internal or external and configured to facilitate coupling with the bottom portion 1012.

In some aspects, the second component 1030 may have the same and/or similar geometry to that of the first component 1010, although the second component may be of an equal or greater size (e.g., length, lateral dimension/diameter, etc.). The second component 1030 is shown to include a substantially cylindrical geometry along the majority of the second component 1030. The first component is further shown to include a top portion 1032 and a bottom portion 1040 which, similar to the second component 1030 as a whole, each include a substantially cylindrical geometry along the majority thereof. In some aspects, the top portion 1032 and the bottom portion 1040 may be releasably couplable with one another (e.g., via an internal threading or other coupling mechanism). Further, in some aspects, the top portion 1032 and the bottom portion 1040 may be integral with one another. Both the top portion 1032 and the bottom portion 1030, when coupled (or integral), are shown to be disposed about a common longitudinal axis (e.g., an axis extending through the cylindrical geometry of both components. In some aspects, one or both of the top portion 1032 and/or the bottom portion 1040 may include a full or partial cannulation extending along some or all of the common longitudinal axis of the components.

The top portion 1032 is shown to include an engagement feature 1034 on an end thereof opposite the bottom portion 1040. The engagement feature 1034 may be configured to interface with one or more of various instruments either via common engagement mechanisms (e.g., tri-lobe, hex-lobe, Phillips, etc.) or via a specialized engagement mechanism configured to facilitate engagement with an instrument specific to the second component 1030. In some aspects, the engagement feature 1034 may have a lesser lateral diameter than other portions of the top portion 1032, although components of the engagement feature 1034 may extend in a lateral direction (e.g., substantially radially) such that a portion of the engagement feature includes a lateral dimension equal to or greater than other portions of the top portion 1032 along the longitudinal axis. Further, in some aspects the engagement feature 1034 may include external threading (which may be partial or intermittent threading, e.g., a threading that does not continue circumferentially about the top portion 1032) or other additional/alternate engagement elements also configured to engage either instruments incorporated in conjunction with the system 1000 or, in some aspects, configured to aid in coupling with the calcaneum 100 and/or the talus 200. The top portion 1032 may also include additional threading (not shown) which may be either internal or external and configured to facilitate coupling with the bottom portion 1040.

The bottom portion 1040, as shown, may include a greater lateral dimension (e.g., diameter) than some or all of the top portion 1032. The bottom portion 1040 is shown to include threading 1042 disposed about at least a portion of an exterior surface thereof, as well as an interface 1044 disposed at an end of the bottom portion 1040 substantially opposite the top portion 1032. The threading 1042 may be the same as and/or similar to threading common to orthopedic implants and/or screws (e.g., considered to interface with various different types/portions of bone, for example those that exist at various portions of the calcaneum 100 and/or the talus 200). The interface 1044 is shown to be set in the end portion of the bottom portion 1040 and, in some aspects, may form a depression that extends substantially into at least a portion of the cylindrical geometry of the bottom portion 1040. The interface 1044 may include an interface common to orthopedic instruments, for example a hex-lobe, torxx, Phillips, or other interface, or the interface 1044 may include an alternate interface configured specifically to interface with one or more instruments specific to the system 1000. Additionally, the bottom portion 1040 may also include additional threading (not shown) which may be either internal or external and configured to facilitate coupling with the top portion 1032.

As shown in FIGS. 7-15, when implanted at least a portion of both the first component 1010 and the second component 1030 are configured to interface with/couple with the calcaneum 100 and the talus 200. In some aspects, the first component 1010 may be configured to couple with a portion of the calcaneum 100 at or near (e.g., at or adjacent the surface of the calcaneum 100 or within the footprint thereof) the middle facet 108 as well as a portion of the talus 200 at or near the talar head 206 and/or talar neck 208. The second component 1020 may be configured to couple with a portion of the calcaneum 100 at or near (e.g., at or adjacent the surface of the calcaneum 100 or within the footprint thereof) the posterior facet 106 as well as a portion of the talus 200 (which may be at or near the talar head 206 and/or talar neck 208, or may be distal relative to the aforementioned anatomical landmarks of the talus 200). In some aspects, one or both of the first and second components 1010, 1030 may be coupled with the calcaneum 100 and talus 200 such that the longitudinal axis of one or both implants is aligned with or in a desired position relative to an identified anatomical axis of the subtalar joint. Prior to implanting any component of the system 1000, one or more axes and/or desired positioned for first and second components 1010, 1030 may be identified using imaging (e.g., CT, etc.) with a bore or other volume then created relative to the axes/desired positions. The components of the system 1000 may then be positioned at least partially within the axes/desired positions such that both the first component 1010 and the second component 1030 are coupled with the calcaneum 100 and the talus 200. In some aspects, the first and second components 1010, 1030 may be releasably coupled prior to implantation, while in other aspects, the top portions 1012, 1032 or the bottom portions 1020, 1040 may be implanted separately and then coupled intraoperatively (e.g., the top portion 1012 is implanted within the talus 200, and then the bottom portion 1020 is threadably coupled with the top portion 1012 and the calcaneum 100 in a single, rotational step.

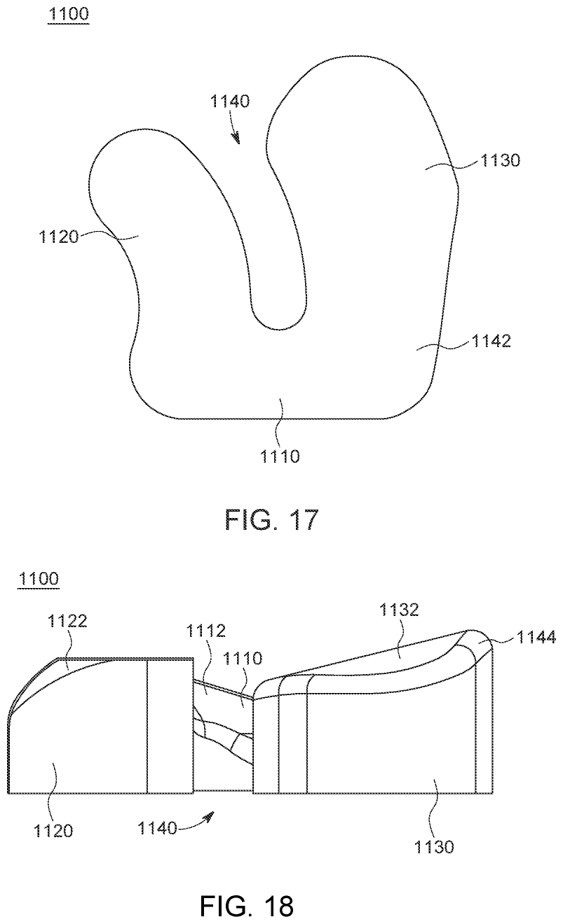

Referring now to FIGS. 16-23, an implant 1100 is shown, according to one aspect of the present disclosure. The implant 1100, as shown, may be a component of an implant system 1000 and also incorporate components of other implant systems including but not limited to those shown and described herein. The implant 1100, as shown, includes a body 1110 including a top surface 1112 of the body 1110. The implant 1100 includes a first leg 1120 having a top surface 1122 and a second leg 1130 having a top surface 1132 extending from the body 1110 and, as show, both the first and second legs 1120, 1130 include substantially rounded terminal ends. In some aspects, the first and second legs 1120, 1130 may extend from the body 1110 in a substantially parallel direction (e.g., so as to for a U-shape) and define a cavity 1040 disposed between the first and second legs 1120, 1130. The implant 1100 is further shown to include a bottom surface 1142 which extends from the body 1110 to both the first leg 1120 and the second leg 1130 and, as shown in FIG. 17, is substantially flat. However, in some aspects, one or more portions of the bottom surface 1142 may include various curvatures, convexities, and/or concavities. In some aspects, the bottom surface 1142 may be configured to interface with a surface of a bone, for example a top surface or a prepared surface of the calcaneum 100. In some aspects, the bottom surface 1142 may include one or more features to facilitate coupling with the calcaneum 100, for example, a surface texture, or a lattice structure to promote bony ingrowth, or one or more apertures/bores/etc. configured to receive a fastener or other coupling component configured to couple the implant 1100 (and/or the first and/or second legs 1120, 1130) with a bone (e.g., the calcaneum 100).

The top surfaces 1112, 1122, and 1132 of the body 1110, the first leg 1120, and the second leg 1130 are shown to be substantially continuous with one another (e.g., have substantially smooth surfaces transitioning from one top surface to another). The implant 1100 is also shown to include a ridge 1144 defining a perimeter of each of the top surfaces 1112, 1122, and 1132. As shown, the ridge 1144 has a substantially convex, rounded, curved geometry which transitions the aforementioned top surfaces to substantially flat lateral surfaces. As shown, the ridge 1144 is continuous about the outer portion of each of the top surfaces, although in some aspects the ridge 1144 may have breaks in order to accommodate interfacing of the implant 1100 with one or more other components of an implant system.

The top surface 1122 of the second leg 1120 is shown to have a concavity extending along at least a portion thereof, where the concavity is pitched such that the concavity slopes away from the cavity 1140 (e.g., toward the outer edge of the implant 1100 and such that the portions of the top surface 1122 closest the cavity 1140 and adjacent the top surface 1112 is at a greater elevation than the portion of the top surface 1122 furthest the cavity 1140). As the top surface 1122 transitions to the top surface 1112 of the body 1110, an edge of the concavity is reached and the top surface 1122 extends to the top surface 1112 which, as shown, is pitched substantially toward the top surface 1132 of the second leg 1130. The top surface 1132 is shown to include a concavity that is pitched such that a portion of the top surface 1132 adjacent the cavity 1140 has a lesser elevation than a portion of the top surface 1132 adjacent a lateral edge of the second leg 1130. Additionally, the top surface 1132 is also pitched such that the portion of the top surface 1132 adjacent the top surface 1112 has a lesser elevation than the portion of the top surface 1132 at a terminal end of the second leg 1130. Accordingly, a point of least elevation (e.g., low point) common to both the top surfaces 1112, 1132 exists at an interface of the top surfaces 1112, 1132 at or adjacent to an interface of the body 1110 and the second leg 1130.

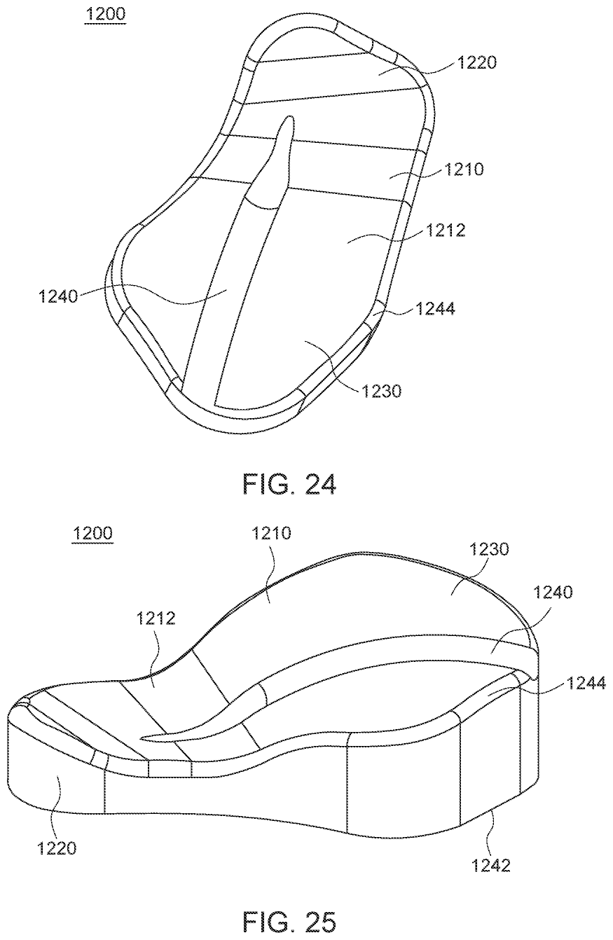

Referring now to FIGS. 24-28, an implant 1200 is shown, according to one aspect of the present disclosure. The implant 1200, as shown, may be a component of an implant system and also incorporate components of other implant systems including, but not limited to those shown and described herein. The implant 1200, as shown, includes a body 1210 including a top surface 1212 of the body 1210 arranged opposite a substantially flat bottom surface 1242 of the implant 1200. In some aspects, the bottom surface 1242 may include a surface texture, a lattice structure to promote bony ingrowth, or one or more apertures/bores/etc. configured to receive a fastener or other coupling component configured to couple the implant 1200 with a bone (e.g., the calcaneum 100). The top surface 1212 of the body 1210 is shown to include a recess 1240 disposed within the body 1210 and extending across at least a portion of the top surface 1212. As shown, the recess 1240 has a substantially u-shaped geometry, although in some aspects, the recess 1240 may have one or more alternate geometries (e.g., V-shaped, rectangular, square cross-sectional geometry, etc.). The implant 1200 is also shown to include a ridge 1244 defining a perimeter of at least a majority of the top surface 1212. As shown, the ridge 1244 has a substantially convex, rounded, curved geometry which transitions the aforementioned top surface 1212 to substantially flat lateral surfaces. The ridge 1244 is substantially continuous about the outer portion of the top surface 1212, except for a single break in the ridge 1244 where the recess 1240 extends to the lateral edge of the top surface 1212 (and thus extends to a lateral surface of the implant 1200). In some aspects, the ridge 1244 may span the entire perimetry of the top surface 1212 (e.g., such that the recess 1240 does not reach a lateral edge of the implant 1200), or may have multiple breaks in order to accommodate the recess 1240 reaching the lateral edge of the implant 1200 at multiple locations or to facilitate interfacing of the implant 1200 with one or more other components of an implant system.

The body 1210 is shown to include a first portion 1220 and a second portion 1230 of the implant 1200. The first portion 1220 is shown to include a lesser vertical dimension than that of the second portion 1230. Further, the recess 1240 is shown to begin/terminate on the top surface 1212 on the first portion 1220, and extend across the top surface 1212 of the first portion 1220 to the second portion 1230 where the recess 1240 interrupts the ridge 1244 and reaches the lateral edge of the implant 1200. The first portion 1220 includes a greater elevation at a point opposite a transition to the second portion 1230 such that the top surface 1212 is pitched substantially downward as it extends from a lateral edge of the first portion 1220 opposite the second portion 1230 toward the interface between the first portion 1220 and the second portion 1230. The recess 1240 begins/terminates at or near a point of the implant 1200 with the least verticality (e.g., shortest in vertical dimension, a concavity) which is positioned on the first portion 1220 adjacent the interface of the first portion 1220 and the second portion 1230. The recess 1240 then follows an incline in the top surface 1212 extending upward on the second portion 1230 until the recess 1240 reaches an apex of the second portion 1230 and subsequently extends to the lateral edge of the top surface 1212. As shown, the second portion 1230 includes a greater vertical dimension than the first portion 1220, with the top surface 1212 being sloped substantially downward (e.g., forming a concavity) from the second portion 1230 down to the first portion 1220. As shown, the top surface 1212 disposed on the second portion 1230 includes a substantially convex geometry which, as shown, includes a substantially rounded geometry at the apex of the vertical dimension of the second portion 1330.

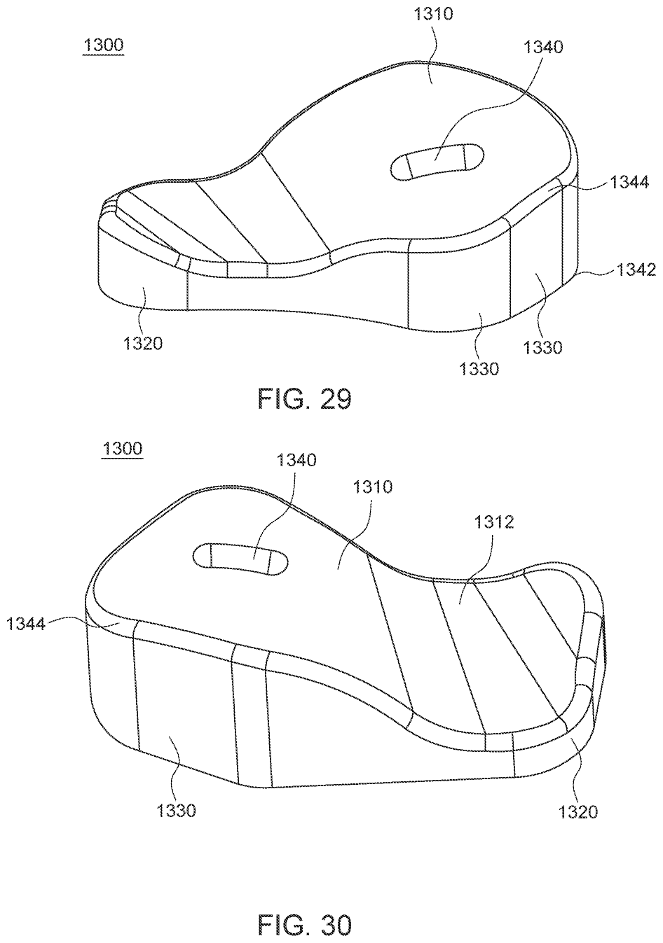

Referring now to FIGS. 29-32, an implant 1300 is shown, according to one aspect of the present disclosure. The implant 1300, as shown, may be a component of an implant system and also incorporate components of other implant systems including but not limited to those shown and described herein. The implant 1300, as shown, includes a body 1310 including a top surface 1312 of the body 1310 arranged opposite a substantially flat bottom surface 1342 of the implant 1300. In some aspects, the bottom surface 1342 may include a surface texture or a lattice structure to promote bony ingrowth, or one or more apertures/bores/etc. configured to receive a fastener or other coupling component configured to couple the implant 1300 with a bone (e.g., the calcaneum 100). The top surface 1312 of the body 1310 is shown to include a recess 1340 disposed within the body 1310 and extending across at least a portion of the top surface 1312. As shown, the recess 1340 has a substantially u-shaped geometry, although in some aspects the recess 1340 may have one or more alternate geometries (e.g., V-shaped, rectangular, square cross-sectional geometry, etc.). The implant 1300 is also shown to include a ridge 1344 defining a perimeter of the top surface 1312. As shown, the ridge 1344 has a substantially convex, rounded, curved geometry which transitions the aforementioned top surface 1312 to substantially flat lateral surfaces. The ridge 1344 is shown to be substantially continuous about the outer portion of the top surface 1312, although in some aspects (other than that shown in FIGS. 29-32) there may be break in the ridge 1344 where the recess 1340 extends to the lateral edge of the top surface 1312 (and thus extends to a lateral surface of the implant 1300). In some aspects, the ridge 1344 may span the entire perimetry of the top surface 1312 (e.g., such that the recess 1340 does not reach a lateral edge of the implant 1300), or may have multiple breaks in order to accommodate the recess 1340 reaching the lateral edge of the implant 1300 at multiple locations or to facilitate interfacing of the implant 1300 with one or more other components of an implant system.

The body 1310 is shown to include a first portion 1320 and a second portion 1330 of the implant 1300. The first portion 1320 is shown to include a lesser vertical dimension than that of the second portion 1330. The first portion 1320 includes a greater elevation at a point opposite a transition to the second portion 1330 such that the top surface 1312 is pitched substantially downward as it extends from a lateral edge of the first portion 1320 opposite the second portion 1330 toward the interface between the first portion 1320 and the second portion 1330. A point of the implant 1300 with the least verticality (e.g., shortest in vertical dimension, a concavity) is positioned on the first portion 1320 adjacent the interface of the first portion 1320 and the second portion 1330. The top surface 1312 then follows an incline of the body 1310 extending upward on the second portion 1330 to an apex of the second portion 1330 and subsequently extends to the lateral edge of the top surface 1312. Further, the recess 1340 is shown to begin and terminate on the top surface 1312 at or near the apex of the second portion 1330. As shown, the second portion 1330 includes a greater vertical dimension than the first portion 1320, with the top surface 1312 being sloped substantially downward (e.g., forming a concavity) from the second portion 1330 down to the first portion 1320. As shown, the top surface 1312 disposed on the second portion 1330 includes a substantially convex geometry which, as shown, includes a substantially rounded geometry at the apex of the vertical dimension of the second portion 1330.

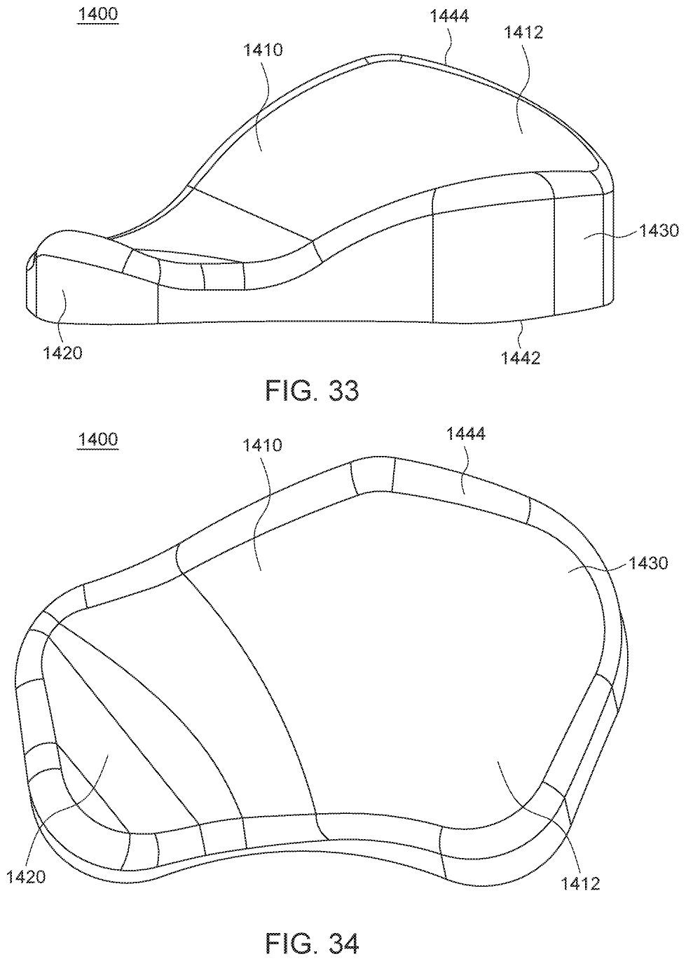

Referring now to FIGS. 33-36, an implant 1400 is shown, according to one aspect of the present disclosure. The implant 1400, as shown, may be a component of an implant system and also incorporate components of other implant systems including but not limited to those shown and described herein. The implant 1400, as shown, includes a body 1410 including a top surface 1412 of the body 1410 arranged opposite a substantially flat bottom surface 1442 of the implant 1400. In some aspects, the bottom surface 1442 may include a surface texture or a lattice structure to promote bony ingrowth, or one or more apertures/bores/etc. configured to receive a fastener or other coupling component configured to couple the implant 1400 with a bone (e.g., the calcaneum 100). The implant 1400 is also shown to include a ridge 1444 defining a perimeter of the top surface 1412. As shown, the ridge 1444 has a substantially convex, rounded, curved geometry which transitions the aforementioned top surface 1412 to substantially flat lateral surfaces. The ridge 1444 is shown to be substantially continuous about the outer portion of the top surface 1412, although in some aspects (other than that shown in FIGS. 33-36), there may be a break in the ridge 1444 where the top surface 1412 extends to the lateral edge of the implant 1400. In some aspects, the ridge 1444 may span the entire perimeter of the top surface 1412 (e.g., such that the top surface 1412 does not reach a lateral edge of the implant 1400), or may have multiple breaks in order to accommodate the top surface 1412 reaching the lateral edge of the implant 1400 at multiple locations or to facilitate interfacing of the implant 1400 with one or more other components of an implant system.

The body 1410 is shown to include a first portion 1420 and a second portion 1430 of the implant 1400. The first portion 1420 is shown to include a lesser vertical dimension than that of the second portion 1430. The first portion 1420 includes a greater elevation at a point opposite a transition to the second portion 1430 such that the top surface 1412 is pitched substantially downward as it extends from a lateral edge of the first portion 1420 opposite the second portion 1430 toward the interface between the first portion 1420 and the second portion 1430. A point of the implant 1400 with the least verticality (e.g., shortest in vertical dimension, a concavity) is positioned on the first portion 1420 adjacent the interface of the first portion 1420 and the second portion 1430. The top surface 1412 then follows an incline of the body 1410 extending upward on the second portion 1430 to an apex of the second portion 1430 and subsequently extends to the lateral edge of the top surface 1412. As shown, the second portion 1430 includes a greater vertical dimension than the first portion 1420, with the top surface 1412 being sloped substantially downward (e.g., forming a concavity) from the second portion 1430 down to the first portion 1420. As shown, the top surface 1412 disposed on the second portion 1430 includes a substantially convex geometry which, as shown, includes a substantially rounded geometry at the apex of the vertical dimension of the second portion 1430.

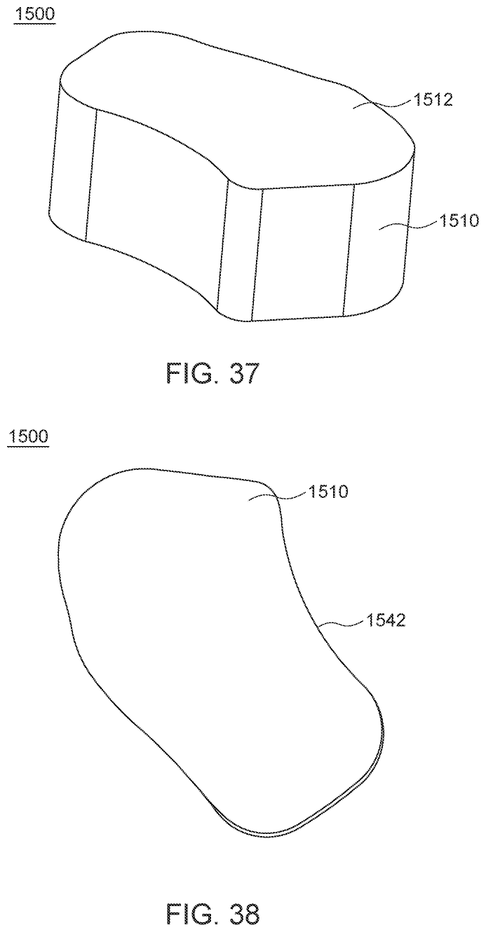

Referring now to FIGS. 37-38, an implant 1500 is shown, according to an exemplary embodiment of the implant system. In some aspects, the implant 1500 may be a component of an implant system and also incorporate components of other implant systems including but not limited to those shown and described herein. The implant 1500, as shown, includes a body 1510 including a top surface 1512 opposite a bottom surface 1542 of the body 1510, where the top and bottom surfaces 1512, 1542 are substantially flat. In some aspects, the top and/or bottom surface 1512, 1542 may be configured to interface with one or more of other implant/implant system components and/or one or more of the calcaneum 100 and the talus 200. In some aspects, the top and/or bottom surfaces 1512, 1542 may include a surface texture or a lattice structure to promote bony ingrowth, or one or more apertures/bores/etc. configured to receive a fastener or other coupling component configured to couple the implant 1500 with a bone (e.g., the calcaneum 100).

It should be understood that one or more of the implants, systems, and/or components thereof as shown and described herein may be incorporated in conjunction with other implants, systems, and or components including but not limited to those shown and described herein as well as those incorporated by reference herein.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. A subtalar joint implant system, comprising:
a first implant component; and
a second implant component.

2. The implant system of claim 1, wherein the first implant component comprises:
a top portion; and
a bottom portion;
wherein the top and bottom portions are coupled with one another and are configured about a common longitudinal axis.

3. The implant system of claim 2, wherein the top portion and the bottom portion of the first implant component are threadably couplable with one another.

4. The implant system of claim 2, wherein the top portion of the first implant component comprises an engagement feature.

5. The implant system of claim 4, wherein the engagement feature comprises an interface which is configured to engage with one or more instruments configured to facilitate implantation of the implant.

6. The implant system of claim 5, wherein the bottom portion comprises:
a threading disposed on at least a portion of an outer surface thereof; and
an engagement feature disposed on an end portion of the bottom portion, wherein the engagement feature is configured to engage with one or more instruments configured to facilitate implantation of the first portion of the implant.

7. The implant system of claim 6, wherein the top portion of the first implant component is configured to be implanted in at least a portion of a talus of a patient.

8. The implant system of claim 7, wherein the bottom portion of the first implant component is configured to be implanted in at least a portion of a calcaneum of the patient.

9. The implant system of claim 8, wherein the bottom portion of the first implant component is configured to be implanted in at least a portion of a posterior facet of the calcaneum of the patient.

10. The implant system of claim 5, wherein the second implant component comprises:
a top portion; and
a bottom portion
wherein the top and bottom portions are coupled with one another and are configured about a common longitudinal axis.

11. The implant system of claim 10, wherein the top portion and the bottom portion of the second implant component are threadably couplable with one another.

12. The implant system of claim 10, wherein the top portion of the second implant component comprises an engagement feature.

13. The implant system of claim 12, wherein the engagement feature comprises an interface which is configured to engage with one or more instruments configured to facilitate implantation of the implant.

14. The implant system of claim 10, wherein the bottom portion comprises:
a threading disposed on at least a portion of an outer surface thereof; and
an engagement feature disposed on an end portion of the bottom portion, wherein the engagement feature is configured to engage with one or more instruments configured to facilitate implantation of the second portion of the implant.

15. The implant system of claim 14, wherein the top portion of the second implant component is configured to be implanted in at least a portion of a talus of a patient.

16. The implant system of claim 15, wherein the bottom portion of the second implant component is configured to be implanted in at least a portion of a calcaneum of the patient.

17. The implant system of claim 16, wherein the bottom portion of the second implant component is configured to be implanted in at least a portion of a middle facet of the calcaneum of the patient.

18. A subtalar implant system, comprising:
a first component, comprising:
a top portion; and
a bottom portion coupled with the top portion and configured about a first common longitudinal axis; and
a second component, comprising:
a top portion; and
a bottom portion coupled with the top portion and configured about a second common longitudinal axis;
wherein the first and second components are each configured to couple with at least a portion of a talus and a calcaneum of a patient.

19. The implant system of claim 18, wherein the first component is configured to couple with at least a portion of a posterior facet of the calcaneum of the patient and the second component is configured to couple with at least a portion of a middle facet of the calcaneum of the patient.

20. A subtalar implant system, comprising:
a first component, comprising a threading disposed on at least a portion of an exterior surface thereof; and
a second component, comprising a threading disposed on at least a portion of an exterior surface thereof;
wherein the first and second components are each configured to couple with at least a portion of a talus and a calcaneum of a patient;
wherein the first component is configured to couple with at least a portion of a posterior facet of the calcaneum of the patient and the second component is configured to couple with at least a portion of a middle facet of the calcaneum of the patient.
